(19) United States
(12) Patent Application Publication
(10) Pub. No.: US 2025/0366828 A1
(43) Pub. Date: Dec. 4, 2025

(54) ULTRASOUND MEASUREMENT INTERFACE SYSTEMS AND METHODS

(71) Applicant: Provisio Medical, Inc., San Diego, CA (US)

(72) Inventor: Stephen Eric Ryan, San Diego, CA (US)

(21) Appl. No.: 18/874,373

(22) PCT Filed: Jun. 28, 2023
(86) PCT No.: PCT/US2023/026465

§ 371 Date: Dec. 12, 2024

Related U.S. Application Data
(60) Provisional application No. 63/367,316, filed on Jun. 29, 2022.

(51) Int. Cl. A61B 8/00; A61B 8/08

(52) U.S. Cl. CPC A61B 8/463; A61B 8/0891; A61B 8/4254; A61B 8/467; A61B 8/5207; A61B 8/5223; A61B 8/5246; A61B 8/5261; A61B 8/5292

(57) ABSTRACT

An interface system for ultrasound measurements, the system including a computer display configured for displaying ultrasound measurements. The system includes one or more processors programmed and configured to receive distance measurements between an ultrasound probe and a lumen wall over different times and longitudinal positions of the lumen wall, each measurement respectively based on an ultrasound signal from a transducer proximate to the lumen wall. Based on the calculated distances, a shape of a cross-section of the lumen is determined. The one or more processors cause the computer display to simultaneously generate a plurality of representations of the lumen indicating differences in size and geometry between the respective shapes.

### REFERENCE TO RELATED APPLICATIONS

This application claims the benefit of U.S. Provisional Application No. 63/367,316, filed Jun. 29, 2022, which is hereby incorporated by reference in its entirety.

### BACKGROUND

### Field

The present disclosure relates generally to systems, methods, and devices that utilize ultrasound to gather dimensional and physiological information about structures such as fluid-filled body vessels.

### Description of the Related Art

Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Thus there is a need for systems, methods, and devices to gather dimensional and physiological information about structures such as fluid-filled body vessels.

### SUMMARY

Embodiments of the present disclosure include novel interfaces and systems for processing and generating displays of ultrasound measurements between an ultrasound probe and a structure (e.g. lumen) wall over different times and longitudinal positions of the structure wall. In some embodiments, the ultrasound measurements include distance [symbol] proximate to the structure wall. The distance measurements may be obtained by analyzing the ultrasound signals for their relative magnitude and time of travel between each respective transducer and the structure wall. Points representing the structure wall and shape determinations (e.g., based on curve-fits) generated therefrom may be based on constructing radial distance lines between the transducer and structure wall from the distance measurements such as described in U.S. Pat. No. 10,231,701 filed Mar. 14, 2014 (the '701 Patent), the entire contents of which is herein incorporated by reference.

The generated shapes may represent cross-sections of a structure (e.g., of a blood vessel wall) at different longitudinal positions within the structure such as by obtaining distance measurements from the ultrasound probe using longitudinally separated radial arrays or by longitudinally re-positioning the probe. In some embodiments, shapes are determined that represent a three-dimensional representation of a structure. The generated curve fits at different longitudinal positions may be graphically represented overlapping each other so as to graphically represent their differences. In some embodiments, overlapping cross-sections pertain to the same relative/approximate section of a structure but relative to different times, such as before and after a treatment is applied to the section (e.g., angioplasty, stenting). In some embodiments, cross-sections and/or three-dimensional representations are displayed adjacent to each other.

The graphical representations of the structure (e.g., curve fits of cross-sectional areas, geometries) and their differences may be presented with calculations of structure dimensions including, for example, diameters, area, and volume of the structure and their differences. The differences in dimensions of cross sections or three-dimensional sections associated with different times may also be calculated and displayed. These differences may illustrate, for example, the differences in the size, shape, and/or condition of an area of the structure before and after treatment so as to show the improvement (or lack thereof) in a treated area.

In some embodiments, the interface provides an option for selecting which areas or portions of a structure to display as overlapping and/or adjacent to each other. A three-dimensional or longitudinal representation may be displayed within which a user may select (e.g., using a mouse or touchscreen cursor) a particular cross-section to also display. In [symbol] representations may be demarked/identified (e.g., highlighted) by a user and stored in memory for later lookup/access/representation.

In some embodiments, graphical representations based on separate measurement and/or imaging modalities may be presented/integrated together. Representations based on the ultrasound distance measurements described herein may be co-registered/aligned and displayed with, for example, angiography, x-ray/fluoroscopy, optical coherence tomography (OCT), and/or intravascular ultrasound (IVUS) imaging data. An interface may be configured to provide a user with an option to select particular regions of the representations for presenting and displaying together so as to provide and compare/augment information from the representations/imaging data in an integrated way to a user. Integrating/displaying the information such as to a physician/technician may better enable them to interpret the information provided from the different measurement/imaging modalities.

In some embodiments, in order to promote a sterile environment, voice commands could be used to interact/manipulate the novel interface and systems. Through use of a trigger word, voice commands could be activated allowing physicians to interact/manipulate the interface and systems without breaking the sterile environment.

In some embodiments, sterile hand signals could be used to promote a sterile environment to interact with the novel interface and systems. In some embodiments, an optical recording device such as a camera integrated into the system could be used to receive hand signals. A trigger hand signal could be used to start each interaction with the system. In some embodiments, a trigger voice command could be used to start each interaction with the system in place of a trigger hand signal. Subsequent hand signals could be used to interact with the interface and system to achieve the desired objective.

Functionality of commands could include going to other interface screens. Additional function commands could include starting, ending, or pausing a run. Switching to different multimodality image views such as OCT to IVUS to Angiogram within the SLT system interface. Switching to three-dimensional SLT view. Bookmarking a spline for further analysis later. Compare command of two bookmarked splines such as a spline bookmarked before treatment and a spline taken after intervention to observe net differences in vessel lumen size or morphology. This list is merely illustrative and is not intended to be limiting. [symbol] includes a computer display configured for displaying the ultrasound measurements and one or more processors programmed and configured to receive sets of ultrasound signals through a plurality of transducers of an ultrasound probe proximate to a lumen wall; for each received ultrasound signal, calculate a distance between the receiving ultrasound transducer and the lumen; based on the calculated distances for each set of ultrasound signals, determine a respective shape of a cross-section of the lumen; and cause the computer display to simultaneously generate a plurality of representations of the lumen indicating differences in size and geometry between the respective shapes.

In some embodiments, the plurality of representations includes two or more of the respective shapes overlapping each other from a front-facing perspective. In some embodiments, each respective shape of a cross-section of the lumen represents a different longitudinal position of the lumen. In some embodiments, a plurality of the respective shapes of a cross-section of the lumen represent a same longitudinal position of the lumen at different time points. In some embodiments, a first time point of the different time points represents a time prior to a lumen-treatment procedure and a second time point of the different time points represents a time after the lumen-treatment procedure. In some embodiments, the lumen-treatment procedure is at least one of a stent placement, angioplasty, or obstruction crossing procedure.

In some embodiments, an interface system for ultrasound measurements includes a computer display configured for displaying the ultrasound measurements and one or more processors programmed and configured to receive sets of ultrasound signals through a plurality of transducers of an ultrasound probe proximate to a lumen wall; for each received ultrasound signal, calculate a distance between the receiving ultrasound transducer and the lumen; based on the calculated distances for each set of ultrasound signals, determine a respective shape of a cross-section of the lumen; receive image data representing the lumen wall, the image data distinct from the calculated distance measurements; and cause the computer display to generate a representation of the respective shapes adjacent a representation of the image data.

In some embodiments, the image data representing the lumen wall comprises at least one of angiography, optical coherence tomography (OCT), or intravascular [symbol] with the respective shapes to co-align with corresponding regions of the structure wall. In some embodiments, a representation of the shapes includes a longitudinal curve fit and where the representation of the image data includes a longitudinal representation spatially co-registered with the longitudinal curve fit. In some embodiments, the one or more processors are further programmed to cause the computer display to present an option for a user to select a longitudinal position within the longitudinal curve fit for generating a corresponding representation of a cross-sectional curve fit and co-registered cross-sectional image data.

In some embodiments, a method for generating images of ultrasound measurements includes receiving distance measurements between a probe and a structure wall over different times and longitudinal positions of the structure wall, each measurement respectively based on an ultrasound signal from a transducer proximate to the structure wall; determining a plurality of curve fits based on the distance measurements, the plurality of the curve fits each representing a cross-section of the structure; and causing the computer display to generate a representation of the plurality of the curve fits overlapping each other and with respect to the different times or longitudinal positions.

In some embodiments, a method for generating images of ultrasound measurements includes receiving sets of ultrasound signals through a plurality of transducers of an ultrasound probe proximate to a lumen wall; for each received ultrasound signal, calculating a distance between the receiving ultrasound transducer and the lumen; based on the calculated distances for each set of ultrasound signals, determining a respective shape of a cross-section of the lumen; and simultaneously generating in a computer display a plurality of representations of the lumen indicating differences in size and geometry between the respective shapes. In some embodiments, the image data representing the lumen wall comprises at least one of angiography, optical coherence tomography (OCT), or intravascular ultrasound (IVUS) image data.

In some embodiments, a method for interacting with the interface system or computer display through gestures and voice commands is disclosed herein. In some embodiments, a user interacts through gestures and voice commands. In some embodiments, use of the voice commands promote a sterile environment. In some embodiments, the voice [symbol] interface systems without breaking the sterile environment.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system with an array of transducers according to some embodiments.

FIG. 2A is an illustrative side perspective diagram of an ultrasound catheter probe placed within a lumen according to some embodiments.

FIG. 2B is a cross-sectional perspective diagram of the ultrasound catheter probe of FIG. 2A.

FIG. 2C is another cross-sectional perspective diagram of the ultrasound catheter probe of FIG. 2A.

FIG. 3 is an illustrative diagram of an interface for representing ultrasound measurements of a structure according to some embodiments.

FIG. 4 is an illustrative diagram of an interface 400 for simultaneously representing longitudinally separate or separately timed sets of ultrasound measurements of a structure according to some embodiments. [symbol] representation of ultrasound measurements of a structure according to some embodiments.

FIG. 6 is an illustrative diagram of an interface for classifying sets of ultrasound measurements of a structure.

FIG. 7 is a block diagram of a process for an interface presenting ultrasound measurements of a structure according to some embodiments.

FIG. 8 is an illustrative diagram of an interface for representing ultrasound measurements of a structure with separate measurement/imaging modalities according to some embodiments.

FIG. 9 is an illustrative diagram of an interface for representing ultrasound measurements of a structure with separate measurement/imaging modalities according to some embodiments.

FIG. 10 is a block diagram of a process for an interface presenting ultrasound measurements co-registered with separate measurement/imaging modalities according to some embodiments.

FIG. 11 is an illustrative diagram of an interface for representing ultrasound measurements of a structure with separate measurement/imaging modalities according to some embodiments.

### DETAILED DESCRIPTION

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have shown that outcomes are significantly improved through the use of more advanced, more accurate imaging techniques.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) [symbol] characterize atherosclerosis and other vessel diseases and defects.

IVUS and OCT images can be used to determine information about a vessel, including vessel dimensions, and are typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure. Some systems are described in which multiple lumen wall distances are measured and a shape of the wall is calculated using the distance measurements such as described in the '701 Patent.

While current IVUS and OCT systems provide additional and more detailed information compared to angiograms, these IVUS and OCT systems introduce significant additional time, cost and complexity into minimally-invasive procedures. Further, the images produced by IVUS, OCT, and angiography systems may not directly provide useful information about blood vessels and are typically subject to nonconforming interpretations of different physicians. Interpretation of IVUS, OCT, and/or angiogram images alone or out of context with more useful information may also not provide physicians with adequate information to select or guide treatment. Thus, there is a need for improved systems for guiding physicians with useful information for guiding and assessing the treatment of patients including, for example, selecting cardiovascular treatments (e.g., angioplasty, stenting, stent coatings), the parameters (e.g., stent size) used for such procedures, evaluation of the outcomes, and whether follow-up treatments may be needed.

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure. It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected [symbol] intervening elements or layers present.

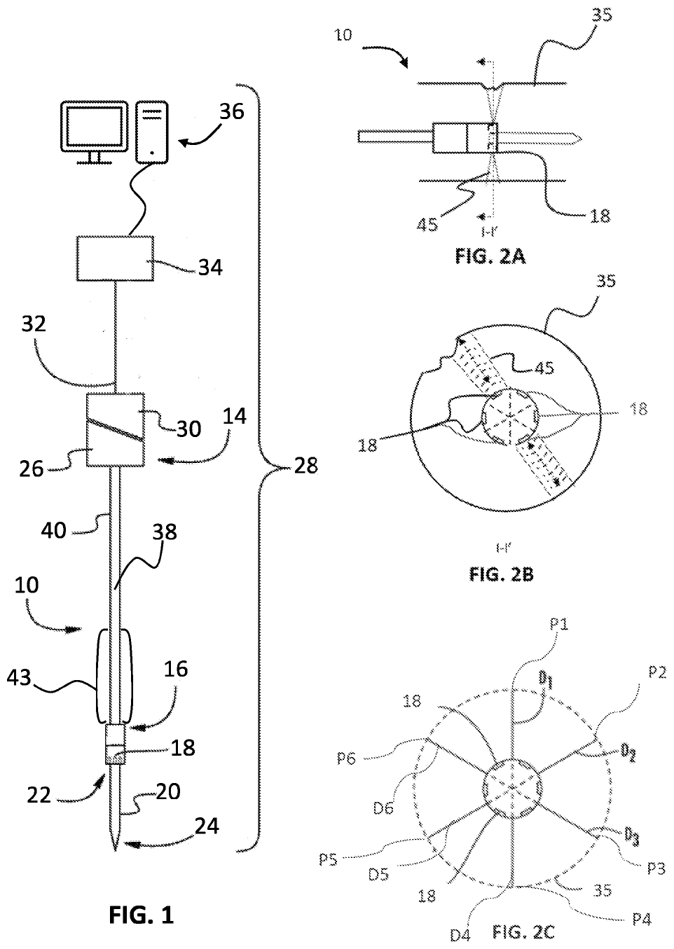

FIG. 1 is an illustrative diagram of an ultrasound catheter probe system 28 with an array of transducers according to some embodiments. In certain embodiments, an ultrasound imaging probe 10 or medical device includes a body 40 having a proximal end 14 and a distal end 16. In certain embodiments, the probe 10 includes a plurality of transducers 18. In certain embodiments, the probe 10 comprises an elongated tip 20 having a proximal end 22 and a distal end 24. In certain embodiments, the probe 10 comprises a proximal connector 26 which connects the probe 10 to other components of the system 28, for example, a data acquisition unit 34 and computer system 36. In certain embodiments, the imaging probe 10 is part of a system 28 that includes a distal connector 30, electrical conductor 32, the data acquisition unit 34, and/or the computer system 36.

In some embodiments, the body 40 is tubular and has a central lumen for containing various connectors and channels that extend toward the distal end 16. In some embodiments, the body 40 has a diameter of about 650 μm or less. These dimensions are illustrative and not intended to be limiting. In some embodiments, the diameter of the probe 10 will depend on the type of device that probe 10 is integrated with and where the probe 10 will be used (e.g., in a blood vessel), which will become apparent to those of ordinary skill in the art in view of the present disclosure.

In certain embodiments, the proximal end 14 of the body 40 can be attached to the proximal connector 26. In some embodiments, the probe 10 includes an elongated tip 20 in which its proximal end 22 is attached to the distal end 16 of the body 40. The elongated tip 20 may be constructed with an appropriate size, strength, and flexibility to be used for guiding the probe 10 through a body lumen (e.g., a blood vessel). In certain embodiments, the elongated tip 20 and/or other components of the probe 10 include one or more radio-markers (e.g., visible to angiography) for precisely guiding the catheter through a lumen and positioning the transducers 18 in the desired location. In some embodiments, the probe 10 and the distal end 16 are constructed and arranged for rapid exchange use. In certain embodiments, the body 40 and the elongated tip 20 are made of resilient flexible biocompatible material such as is common for IVUS and intravascular catheters known to those of ordinary skill in the art. [symbol] central lumen 38. In certain embodiments, a lumen-expanding balloon 43 surrounds and is integrated with the body 40 and are used for treatment (e.g., angioplasty, stenting) and/or holding the probe 10 in place while performing measurements. In some embodiments, the probe 10 may have lumens for use with various features not shown (guidewires, fiberoptics, saline flush lumens, electrical connectors, etc.). In some embodiments, the outer diameter of the body 40 and the elongated tip 20, if present, is substantially consistent along its length and does not exceed a predetermined amount.

In certain embodiments, the transducers 18 may be incorporated with the body 40 of the distal end 16 such as described further herein so as to reduce the footprint of the body 40. In certain embodiments, the transducers 18 are connected by one or more conductors extending through the lumen 38 to a data acquisition unit 34. In certain embodiments, signals received and processed by the data acquisition unit 34 are then processed by the computer system 36 programmed to store and analyze the signals (e.g., calculate distance measurements between the catheter and lumen wall). In some embodiments, by reducing the footprint of the body 40, the space saved is utilized to incorporate additional features (e.g., an expandable balloon 43, balloon media lumen for expanding and deflating balloon 43). In some embodiments, the area of the face of each transducer's piezoelectric layer is at least about 2500 square microns and/or has a width of about 50 microns or more.

In some embodiments, the ultrasound transducers 18 are piezoelectric. The transducers may be built using piezoelectric ceramic or crystal material; as well as piezoelectric composites of ceramic or crystal material with epoxies. In some embodiments, the transducers use piezoelectric crystals composed of Pb(Mg_{1/3}Nb_{2/3})O₃—PbTiO3 (PMN-PT) or other types of piezoelectric materials with dimensions configured to resonate, for example, at predetermined frequencies. In some embodiments, the transducers are photoacoustic transducers and/or ultrasonic sensors that use MEMS (Microelectromechanical Systems) technology, such as but not limited to PMUTs (Piezoelectric Micromachined Ultrasonic Transducers) and CMUTs (Capacitive Micromachined Ultrasonic Transducers).

In certain embodiments, the operating frequency for the ultrasound transducers is in the range of about 8 to about 50 MHz or even up to about 60 MHz, depending application.

Generally, higher frequency of operation provides better resolution and a smaller device 10. However, the tradeoff for this higher resolution and smaller catheter size may be a reduced depth of penetration into the tissue of interest and an increase in echoes from the blood itself (making the image more difficult to interpret). Lower frequency operation may be more suitable for imaging in larger vessels or within structures such as the chambers of the heart. Although specific frequency ranges have been given, these ranges given are illustrative and not limiting. The ultrasonic transducers 18 may produce and receive any frequency that leaves a transducer 18, impinges on some structure or material of interest and is reflected back to and picked up by a transducer 18.

The center resonant frequency and bandwidth of a transducer is generally related to the thickness of transducer materials generating or responding to ultrasound signals. For example, in some embodiments, a transducer includes a piezoelectric material such as quartz and/or lead-zirconate-titanate (PZT). A thicker layer will generally respond to a longer wavelength and lower frequency and vice versa. For example, a 50 micron thick layer of PZT will have a resonant frequency of about 40 MHz, a 65 micron thick layer will have a resonant frequency of about 30 MHz, and a 100 micron layer will have a resonant frequency of about 20 MHz. As further described herein, matching and backing layers may be included, reduced, or omitted which affect the bandwidth and other characteristics of a transducer.

In some embodiments, a resonant frequency of some transducers is centered around 20, 25, or 30 MHz while other transducers have a resonant frequency centered around 35, 40, 45, or 50 MHz, for example. The respective materials and dimensions of the transducer layers may be configured accordingly. Subsets of transducers may be activated at the same time while other subsets activated at a separate time. In some embodiments, an electronic switch is utilized to switch connections between different transducers or subsets of transducers.

In some embodiments, the probe 10 is connected with an actuating mechanism that rotates and/or longitudinally moves at least some portions of the probe 10 and its transducers 18. A controlled longitudinal and/or radial movement permits the probe to obtain ultrasound readings from different perspectives within a surrounding structure, for example. In certain embodiments, positioning the probe and its transducers in target locations [symbol] system 28. Relative positions of the probe may be tracked and recorded during such processes (e.g., by using an encoder or other position sensing tool).

In some embodiments, the system 28 is programmed to analyze and identify characteristics of the medium (e.g., blood) between the probe 10 and structure in order to determine where the medium ends with respect to the structure (e.g., blood vessel wall). In some embodiments, multiple ultrasound images of the blood are generated and the differences between the images are used to identify movement/change of the blood over time (e.g., as a result of a heart pumping). In some embodiments, doppler echo signals are used to determine these differences. Because the blood vessel wall does not have the same movement/change characteristics as the blood, the amount (or distance) between the probe 10 and blood vessel wall can be calculated. In some cases, reliance on the blood images without substantial reliance on images of the blood vessel wall is used to determine the distance between the probe 10 and blood vessel wall.

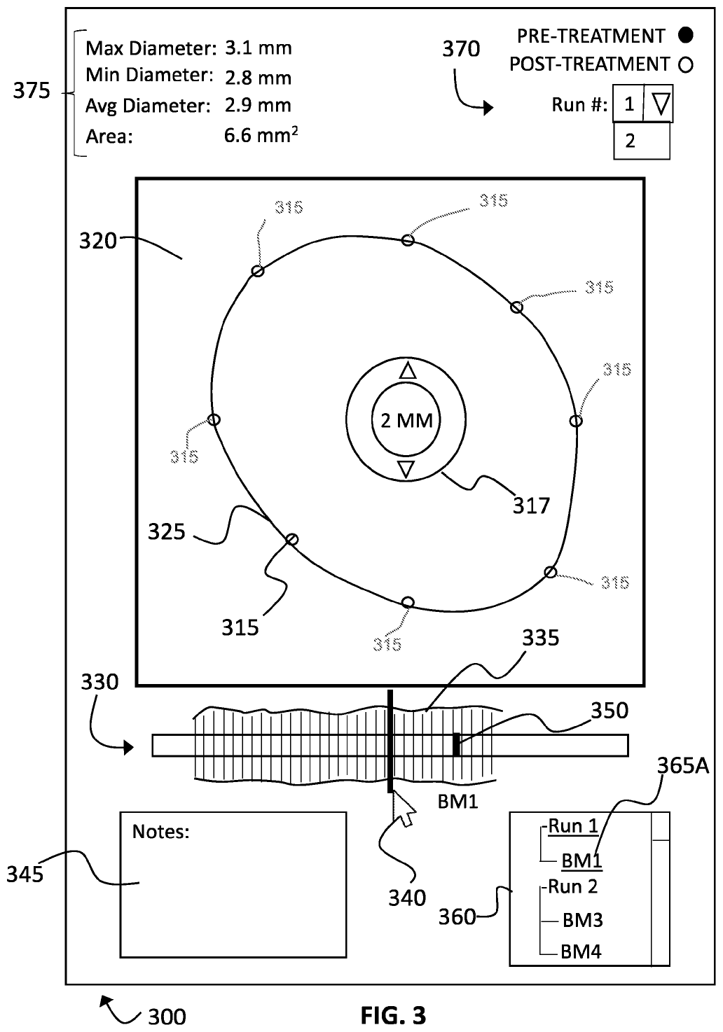

FIG. 2A is an illustrative side perspective diagram of an ultrasound catheter probe placed within a lumen according to some embodiments. FIG. 2B is a cross-sectional perspective diagram of the ultrasound catheter probe across lines I-I′ of FIG. 2A. FIG. 2C is another cross-sectional perspective diagram of the ultrasound catheter probe 10 across lines I-I′ of FIG. 2A. A catheter probe 10 is shown inserted into a lumen 35. A connected computer system (e.g. 36) is programmed to cause transducers 18 (e.g. one or more) to generate pulses 45 where each of the pulses is incident on different portions of the lumen 35. In response to echoes from the lumen 35, the transducers 18 generate electromagnetic signals respective to the first and second pulses that reflect back from media (e.g. blood) and the lumen 35 adjacent probe 10. These electromagnetic signals are then processed by a signal processor and the computer system 36.

The computer system 36 is programmed to analyze and distinguish pertinent imaging data within the frequency response received by the transducers 18. Because the transducers 18 may be configured and arranged with a reduced footprint, including reduced and/or omitted backing and matching layers, the signals associated with imaging data may be obscured by additional noise associated with the activating pulse. In some embodiments, an envelope signal associated with the activating pulse is detected and distinguished within the [symbol] distinction, a distance measurement may be calculated between the transducer/probe and the transition location.

Other pulses may be similarly delivered/echoed using other transducers 18. In some embodiments, these pulses may be delivered simultaneously or at different times. Along with identifying and associating the signals with respective transducers, the computer system 36 is programmed to analyze the signals and calculate a radial distance measurement (e.g. D1, D2, . . . , D6) between each transducer 18 and lumen 35. This may be done, for example, by utilizing time-of-flight information of the echo signals and previously determined/differentiated signatures representative of a lumen wall (e.g., of lumen 35) and a particular medium (e.g., blood) between the transducer 18 and lumen 35. Exemplary systems and methods for making such calculations are described, for example, in the '701 Patent.

Based on distance calculations (D1, D2, . . . , D6), the shape and dimensions of the lumen 35 may be estimated by further utilizing information including the dimensions of the probe 10 and applying interpolation and/or other mathematical fitting techniques. For example, in certain embodiments, the relative positions of points (p1, . . . , p6) about the lumen 35 are first calculated and a curve fitting algorithm (e.g., spline interpolation) is applied to generate a two-dimensional slice representation of the lumen 35. As described in the '701 Patent, multiple slices can be calculated by taking sets of ultrasound readings along the longitudinal extent of lumen 35 and combining them to generate a three-dimensional representation.

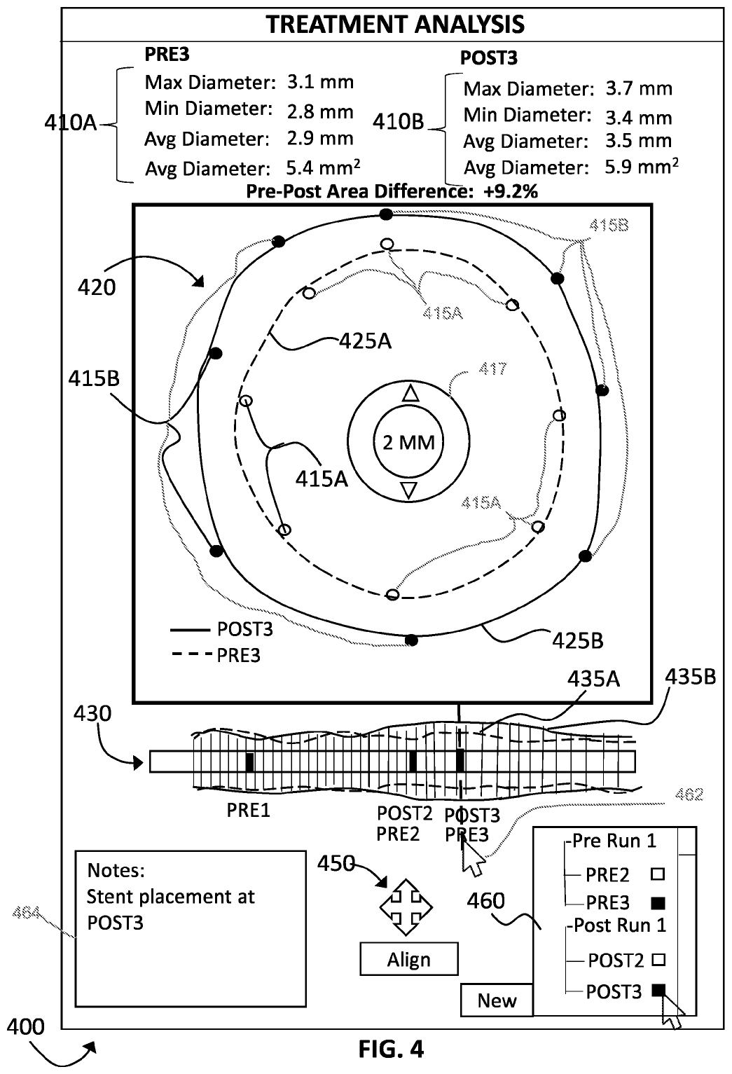

FIG. 3 is an illustrative diagram of an interface 300 for representing ultrasound measurements of a structure according to some embodiments. Interface 300 displays a graphical representation 320 of a lumen cross-section based on distance points 315 calculated from sets of ultrasound signals through a plurality of transducers arranged about a probe 10 (e.g., as shown in FIGS. 1, and 2A-2C). The certain embodiments, distance points are based on time of flight data and on determining the end points of radial distance lines along perpendiculars between receiving transducers and the lumen wall. In some embodiments, after ultrasound measurements are performed, data and/or calculations pertaining to the measurements are stored within a computer storage medium (e.g., a cloud) and accessed by the interface. [symbol] to distance points 315 (e.g., using splines) for estimating a shape of the cross-section. In some embodiments, the representation includes a scale legend 317 indicating the distance across the display relative to a physical distance in units of measure (e.g., millimeters, inches) across the shape/curve 325 and distance points 315. In some embodiments, the scaling is based, at least in part, on the distance measurements used to calculate distance points 315. At 375, various calculated metrics pertaining to the estimated shape of the lumen cross section are displayed. These metrics may include calculations of a maximum diameter, minimum diameter, average diameter, area, and/or other metrics of the cross section.

In some embodiments, the interface 300 provides a timeline display 330 for a user to select a cross-section from among multiple cross-sections and longitudinal positions of a lumen to display. In some embodiments, the timeline display 330 may operate as a sliding scrollbar in which a two-dimensional side-view overlay 335 of the lumen is presented. The overlay 335 may indicate a relative maximum, minimum, or average diameter measured of lumen cross sections at different longitudinal positions. By selecting a particular longitudinal position of the lumen, such as by positioning of a scrollbar using a pointer 340, the corresponding representation 320 of a lumen cross-section is displayed. The timeline display 330 may include identifiers, such as shown at 350, that demark particular longitudinal positions within the lumen. In some embodiments, the identifiers associated with particular positions can be set and saved by a user and listed/selected in a list display 360. The identifier 365A of the presently displayed cross-section is shown highlighted in list display 360.

Separate ultrasound measurement runs of a lumen (e.g., taken before and after a treatment procedure) can also be stored and later accessed by interface 300. A user may set/select a particular run using a selector 370. In some embodiments, upon selecting a run, timeline display 330 is updated with a new longitudinal lumen overlay 335 representing available cross-sections of the lumen that were measured during the run. In some embodiments, in a notes field 345, an operator can enter and store custom notes/observations about a particular run and/or measured cross-section of a lumen. Runs may also be stored by groups (e.g., by pre-treatment runs, post-treatment runs) and identified in sequence within their particular grouping as shown. [symbol] representing longitudinally and/or temporally separate sets of ultrasound measurements of a structure according to some embodiments. In some embodiments, interface 400 displays a graphical representation 420 of two or more lumen cross-sections based on respective distance points calculated from sets of ultrasound signals through a plurality of transducers arranged about a probe 10 (e.g., as shown in FIGS. 1, and 2A-2C). In some embodiments, overlapping cross sections are simultaneously shown, for example, based on sets 415A and 415B of distance points, and based on which shapes (e.g., curve fits) 425A and 425B are respectively generated and represented. In some embodiments, the sets of distance points were obtained from measurements of the same section of a lumen before and after a treatment (e.g., angioplasty/stenting) was performed. In some embodiments, sets of distance points and/or shapes that were measured at separate longitudinal positions of a lumen (e.g., a normal vs. narrowed portion of a lumen) are displayed simultaneously. In some embodiments, the calculated distance points (e.g., 415A,B) are not displayed with the cross-sections/shapes and/or vice versa. In some embodiments, the representation includes a scale legend 417 such as described in FIG. 3.

Calculated metrics of each of the displayed cross-sections are shown at 410A and 410B as well as comparison metrics (e.g., differences between the cross-sections). For example, an operator can compare the areas or diameters of the same longitudinal position of a lumen cross-section before and after a lumen expanding or obstruction crossing procedure (e.g., angioplasty, stenting).

In some embodiments, interface 400 provides a timeline display 430 for a user to select a cross-section from among multiple cross-sections and longitudinal positions of a lumen for display. This may be done by selecting a particular longitudinal position of the lumen using a pointer 462 or by positioning of a scrollbar as described in FIG. 3. some embodiments, the timeline display 430 may operate with a sliding scrollbar in which side-view overlays 435A and 435B of the lumen is presented. The overlays 435A and 435B may indicate comparative maximums, minimums, or average diameters measured of the lumen cross sections at different longitudinal positions and/or time points by selecting a particular longitudinal position of the lumen, such as by positioning of a scrollbar to select a particular position. In some embodiments, the timeline display 430 may include highlighted markers that [symbol] These markers/positions can be set and saved by a user and listed/selected in a list display 460.

In some embodiments, two or more cross-sections may be presented simultaneously. For example, the interface can be arranged to display overlapping cross-sections from two or more different longitudinal positions taken at two or more different time points (e.g., pre-and post-treatment), thereby presenting four different cross-sections simultaneously.

In order to correspond/co-register cross-sections of the same longitudinal position of a lumen with respect to each other, a tool 450 may be used to reposition, scale, and/or align measurements/representations of the same luminal segments (e.g., upon which overlays 435A and 435B are based) with respect to each other. In some embodiments, analysis of the features of the segments measured/imaged at different times (e.g., pre-/post-treatment) are used to co-align the longitudinal segments (e.g., using machine learning/pattern matching). In some embodiments, a tracking process/feature (e.g., a radio-marker) is used to track the position of an imaging probe obtaining measurements and store the tracking information in computer memory and used later to align segment measurements/shapes. In some embodiments, a notes field 464 is used to store custom notes/observations about a particular run and/or measured cross-section of a lumen.

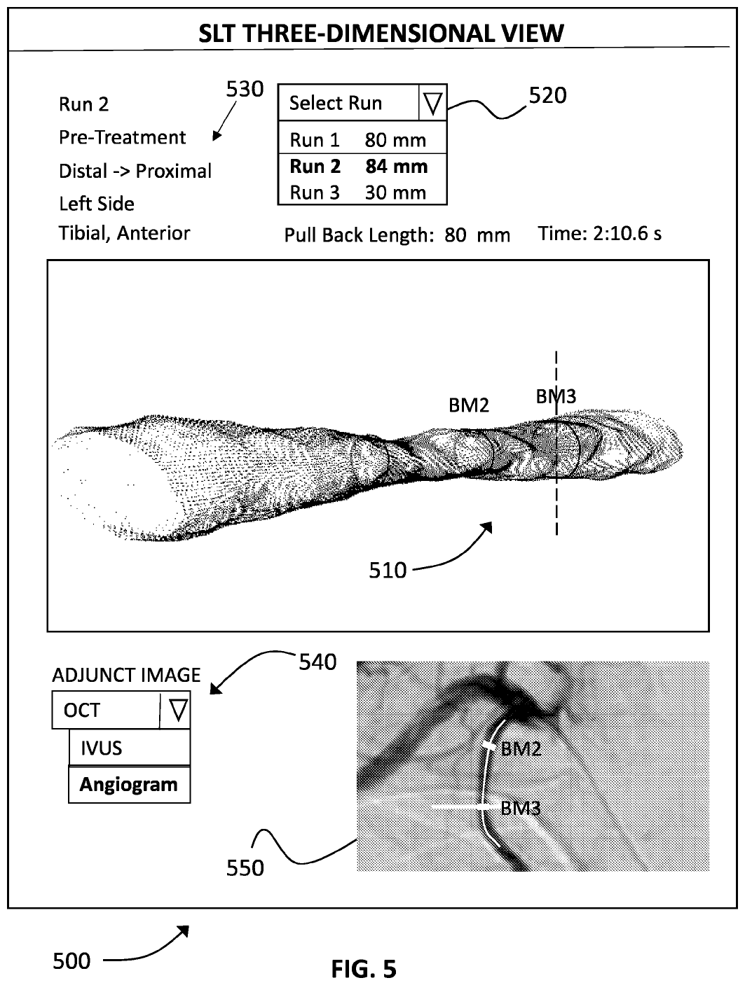

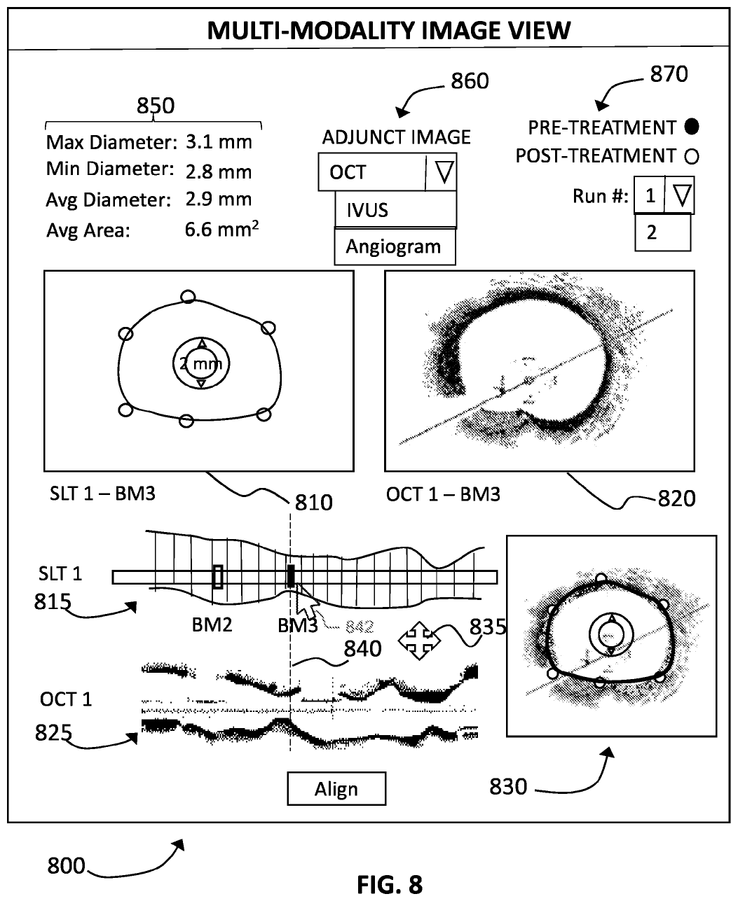

FIG. 5 is an illustrative diagram of an interface 500 for a three-dimensional representation of ultrasound measurements of a structure according to some embodiments. In some embodiments, after multiple cross-sections of a lumen are measured using an ultrasound probe as described herein, sets of measured distance points and/or calculated cross-sectional shapes are used to generate a three-dimensional representation 510 of the lumen based on the points/shapes (e.g., as shown in FIG. 3 or FIG. 4). In some embodiments, curve fits can be calculated to interpolate between measured distance points and shapes at different longitudinal sections of a lumen. In some embodiments, a selector tool 520 may select from among multiple sets of cross-sectional shapes measured during a particular time frame (e.g., a run). In some embodiments, attributes/metrics associated with a three-dimensional representation are shown at 530 and can include, for example, run type, run distance, run direction, run location, entry point, elapsed time of the run, volume, min/max-diameter of the three-dimensional representation. [symbol] measured relative movements of the ultrasound probe (e.g., using a mechanical insertion/pullback mechanism that tracks the movement of the probe between positions). In some embodiments, a separate imaging modality (e.g., angiogram) is selected for co-display using selector 540 and an image of the modality displayed at 550. A user may select which modalities to present at 540. In order to co-register/align three-dimensional ultrasound measurement images and separate imaging modalities, a feature may be integrated with the catheter (e.g., a radio-marker) and used to track the longitudinal position of the probe within the separate imaging system in coordination with the probe obtaining ultrasound distance measurements. In certain embodiments, aligning separate images is performed manually such as further described herein (e.g., with respect to FIGS. 4 and 8).

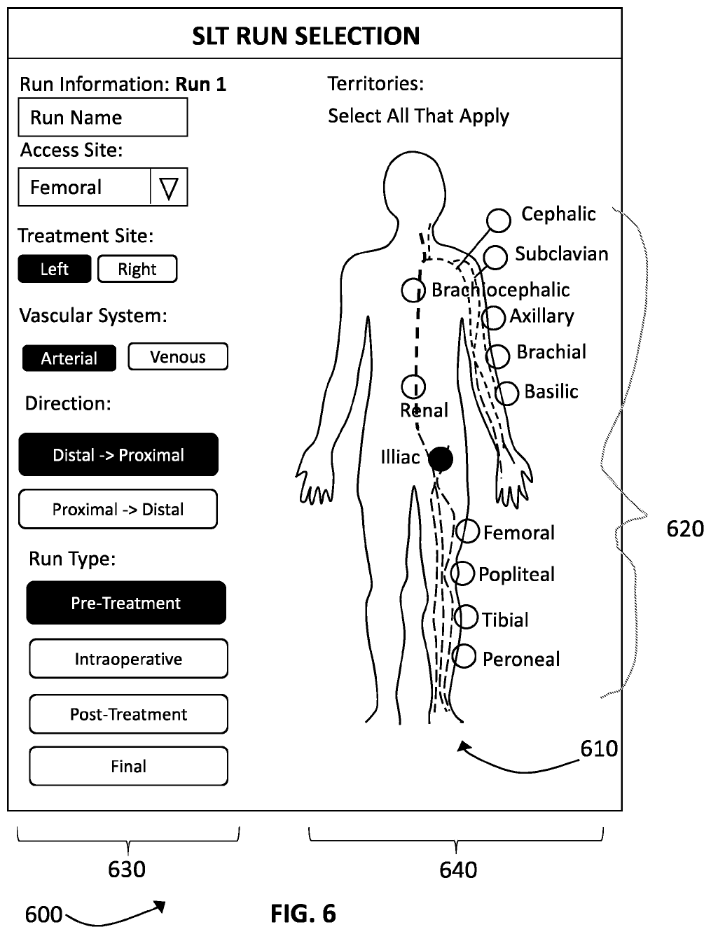

FIG. 6 is an illustrative diagram of an interface for classifying sets of ultrasound measurements of a structure. In some embodiments, an interface 600 provides a selection tool for classifying, storing, and identifying runs of ultrasound measurements in a lumen/body and associated data (e.g., calculated shapes, metrics). At 630, a user may select among various identifying characteristics of one or more runs. A run may be a set of cross-sectional ultrasound measurements taken over the segment of a lumen over a closely proximate series of time points. Information/characteristics identifying separate runs may include a particular treatment site (e.g. left, right), vascular system (e.g. arterial, venous), access cite (e.g. femoral), direction (e.g. distal to proximal, proximal to distal), run type (e.g. pre-treatment, intraoperative, post-treatment, final etc.), and sequence number, for example. At 640, a user may identify a run location using a graphical representation of anatomic territories 610 and selectable options 620 (e.g. cephalic, subclavian, brachiocephalic, axillary, brachial, basilic, renal, iliac, femoral, popliteal, tibial, peroneal etc.) of which iliac is shown selected.

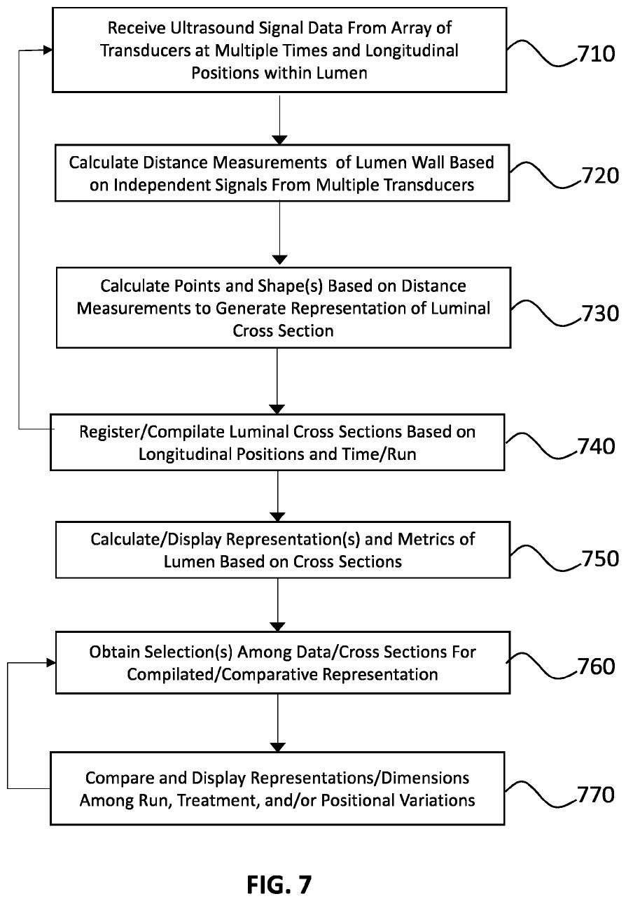

FIG. 7 is a block diagram of a process for an interface presenting ultrasound measurements of a structure according to some embodiments. At block 710, after a probe is inserted into a structure (e.g., a lumen), ultrasound signals are transmitted from and received at a plurality of ultrasound transducers arranged about an ultrasound probe (e.g., probe 10 of FIG. 1). At block 720, based on the signals received at transducers of the probe, distance [symbol] wall).

At block 730, based on the distance measurements and respective perpendiculars from the transducers, points are determined that estimate boundaries of the structure. Using these points, cross-sectional curve fits/shapes of the structure boundary (e.g., lumen wall) are calculated (e.g., as shown in FIG. 3). These curve-fits/shapes may be based on splines, for example.

At block 740, the longitudinal position of the calculated cross-section is tracked (e.g., stored in computer memory). Such as described above, the relative longitudinal positions of the cross-sections may be based on measured relative movements of the ultrasound probe. In some embodiments, a separate imaging modality (e.g., fluoroscopy) may be used to track the longitudinal position of the probe within a lumen in coordination with the probe obtaining ultrasound measurements. After storing a longitudinal cross-section location, additional cross-sections of the structure/lumen are measured starting at block 710. The probe may be moved to a new longitudinal position such as by mechanical actuation so as to position the probe to obtain additional cross-sections at different longitudinal positions of the structure/lumen.

At block 750, after measurements/shapes of one or more cross-sections of the structure are obtained, graphical representations of the cross-section(s) based on the measurements/shapes are generated (e.g., as shown in FIGS. 3, 4, and 5). In addition to generating graphical representations, metrics pertaining to the cross-sections are also calculated/displayed. In some embodiments, a broad view of all of the one or more available cross-section(s) (e.g., a 3D-representation as shown in FIG. 5, also timeline display 330, 430 of FIGS. 3-4) is presented in which a user can select particular cross-section(s).

At block 760, a selection and/or compilation of cross-section(s) is obtained for generating a display of the selection/compilation. In some embodiments, a user may select from the data/cross-sections (e.g. based on a selection process referred to at block 750). In some embodiments, a selection/compilation is based on an analysis of the data/cross-sections. In some embodiments, the analysis includes automatically identifying sections of the lumen that represent possible areas of interest (e.g., narrowing/blockages) in the lumen such as by analyzing/comparing the diameters of cross-sections obtained. [symbol] multiple cross-sections are rendered and displayed. In some embodiments, two or more cross-sections may be presented simultaneously. In some embodiments, the representations show overlapping cross-sections illustrating their relative shape and size differences (e.g., as shown in FIG. 4). In some embodiments, a three-dimensional representation of the lumen is displayed based on a compilation of multiple cross-sections and interpolation therebetween (e.g., as shown in FIG. 5).

FIG. 8 is an illustrative diagram of an interface 800 for representing ultrasound measurements of a structure with separate measurement/imaging modalities according to some embodiments. In some embodiments, the interface 800 provides a representation at 810 of a longitudinal lumen cross-section based on distance points measured by an ultrasound probe such as described herein. In some embodiments, the interface 800 provides a second representation of the same lumen cross-section at 820 of imaging data obtained from a separate modality (i.e., optical coherence tomography (OCT)). Segments of the lumen based on each of the modalities is shown at 815 and 825, respectively. In some embodiments, a user may use a selector 842 and/or a scroll-bar type tool 840 to select a portion of the lumen for which to present corresponding cross-sections/representation at 810 and 820. Calculated metrics of each of the displayed cross-sections are shown at 850. In some embodiments, a user may select which modalities to present at 860 together in the display interface 800. In some embodiments, a user may select at 870 which measurement/imaging runs (e.g., pre-or post-treatment) pertaining to modalities should be presented. In some embodiments, the representations may be presented to overlap each other (e.g., similar to FIG. 4) and metrics pertaining to the selected section of the lumen are displayed at 830.

In order to correspond/co-register cross-sections of different modalities to each other, a tool 835 may be used to reposition, scale, and/or align each of the segments with respect to each other. In some embodiments, analysis of the features of the segments measured/imaged using the separate modalities is used to co-align the longitudinal segments (e.g., using machine learning/pattern matching). In some embodiments, different modalities are spatially correlated/co-registered when the data/images are obtained at the same time and of the same location (e.g., ultrasound-based distance measurements obtained while fluoroscopy was performed). In some embodiments, a tracking process/feature (e.g., a [symbol] store the tracking information in computer memory.

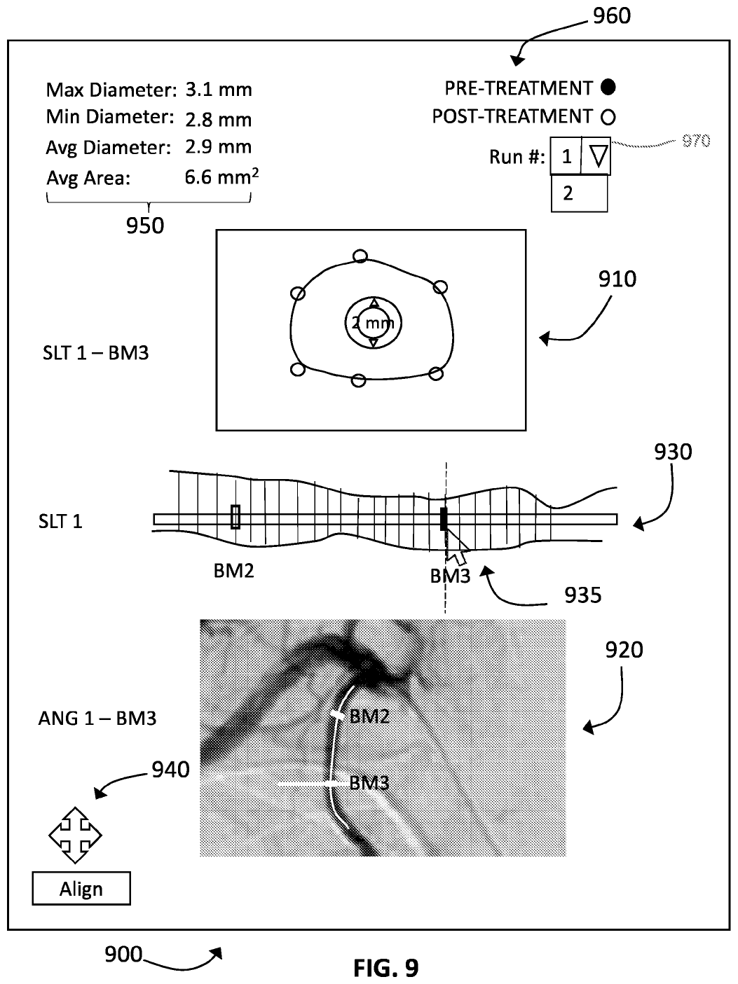

FIG. 9 is an illustrative diagram of an interface 900 for representing ultrasound measurements of a structure with separate measurement/imaging modalities according to some embodiments. In some embodiments, the interface 900 provides a representation at 910 of a lumen cross-section based on distance points measured by an ultrasound probe such as described herein. In some embodiments, a representation of a longitudinal segment is generated at 930 based on the different sets of distance measurements obtained by the probe at separate longitudinal positions within the lumen. A radiographic (e.g., angiogram/fluoroscopy) image is also generated at 920 from data obtained of the same area where the ultrasound probe obtained its distance measurements. In some embodiments, the position of the probe within the lumen and in the radiographic image may be tracked in time and position such as by using a radio-marker integrated with the probe as described herein.

Overlays within the radiographic image at 920 may include highlighted markers (e.g. BM2, BM3) correlating positions in the image with longitudinal positions shown at 930 for the ultrasound-based distance measurements. A user may operate a pointer 935 to select a cross-section to display at 910 by selecting particular portions of the lumen within the segment shown at 930 and/or radiographic image at 920 and by using a sliding bar as described herein. A user may select which run among multiple measurement runs to select for display at 970. The corresponding longitudinal luminal positions shown in the radiographic image at 920 and segment at 910 can be aligned manually or automatically such as described herein such as by using a manual tool 940 or automated alignment. Calculated metrics of each of the displayed cross-sections are shown at 950. In some embodiments, a user may select at 960 which measurement/imaging runs (e.g., pre-or post-treatment) pertaining to modalities should be presented.

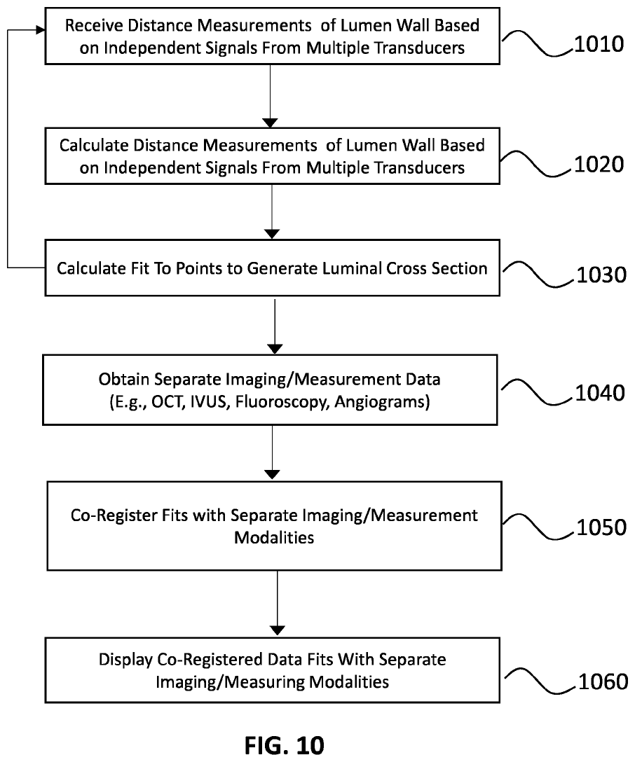

FIG. 10 is a block diagram of a process for an interface presenting ultrasound measurements co-registered with separate measurement/imaging modalities according to some embodiments. At block 1010, a probe having a plurality of ultrasound transducers (e.g., probe 10 of FIG. 1) transmits and receives signals to and from a structure (e.g., a lumen wall). At block 1020, based on the signals received at transducers of the probe, distance measurements are calculated between the transducers and the adjacent structure. At [symbol] structure are calculated with respect to the probe (e.g., based on perpendiculars between the transducers and structure wall), and a curve/shape-fit to the wall boundary is further calculated that represents a cross-sectional map/virtual image of the lumen (e.g., as illustrated in FIGS. 3, 4, 8, and 9).

At block 1040, one or more imaging/measurement modalities pertaining to the structure which are at least partially separate from the distance measurements which are obtained at block 1010. In some embodiments, these modalities may include one or more of optical coherence tomography (OCT), ultrasound (e.g., intravascular ultrasound (IVUS)), x-ray (e.g., fluoroscopy, angiography, computed tomography (CT) scanning), magnetic resonance imaging (MRI), functional MRI (fMRI), nuclear medicine imaging, positron-emission tomography (PET), spectroscopy (e.g., near infrared spectroscopy), and numerous others that are known to one of ordinary skill in the art.

At block 1050, the one or more modalities are co-aligned/registered with the calculated shapes/distance measurements obtained at blocks 1010 to 1030. As further described herein, co-aligning/registering may be performed based on automated coordinating/correlating of data/timing/positioning of the modalities with the distance measurements/shapes and/or by manual alignment through an operator. At block 1060, the calculated shapes/distance measurements and the other modalities obtained at block 1040 are graphically represented in a computer interface (e.g., as shown in FIGS. 8 and 9). Metrics based on the calculated shapes/distance measurements and other modalities may also be presented in the interface and may include the results/calculations of comparative analysis across/between them.

The processes described herein (e.g., the processes of FIGS. 7 and 10) are not limited to use with the hardware shown and described herein. They may find applicability in any computing or processing environment and with any type of machine or set of machines that is capable of running a computer program. In certain embodiments, the processes described herein are implemented in hardware, software, or a combination of the two. In certain embodiments, the processes described herein are implemented in computer programs executed on programmable computers/machines that each includes a processor, a non-transitory machine-readable medium or other article of manufacture that is readable by the processor [symbol] and one or more output devices. In certain embodiments, program code is applied to data entered using an input device to perform any of the processes described herein and to generate output information.

In certain embodiments, the processing blocks (for example, in the processes of FIGS. 7 and 10) associated with implementing the system are performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

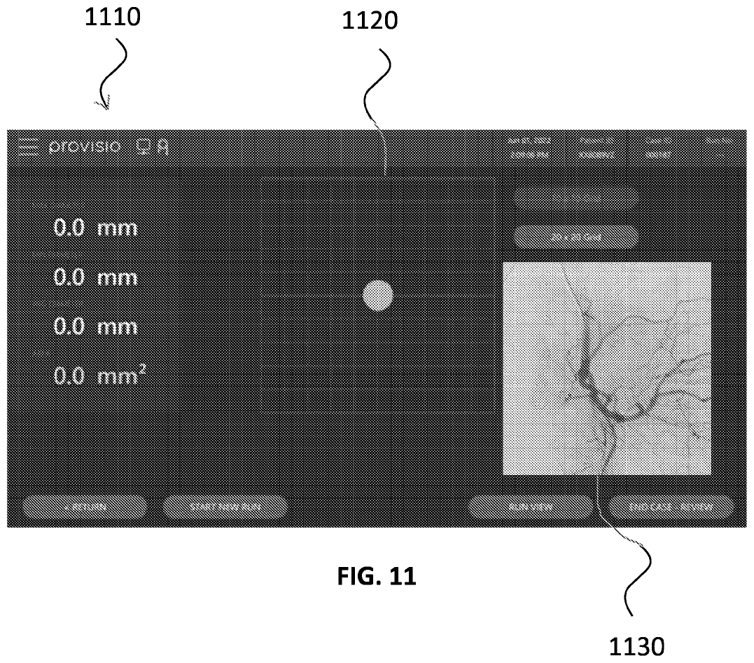

FIG. 11 is another illustrative diagram of an interface 1110 for representing ultrasound measurements of a structure with separate measurement/imaging modalities according to some embodiments. In some embodiments, the interface 1110 provides a representation at 1120 of a lumen cross-section based on distance points measured by an ultrasound probe such as described herein. A radiographic (e.g., angiogram/fluoroscopy) image 1130 is also generated from data obtained of the same area where the ultrasound probe obtained its distance measurements. In some embodiments, the position of the probe within the lumen and in the radiographic image may be tracked in time and position such as by using a radio-marker integrated with the probe as described herein. In some embodiments, the position of the probe within the lumen may be recorded allowing for further analysis of vessel morphology with longitudinal positioning of the probe within the vessel.

The processes described herein are not limited to the specific examples described. For example, the processes of FIGS. 7 and 10 are not limited to the specific processing orders illustrated. Rather, any of the processing blocks of FIGS. 7 and 10 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any [symbol] the scope of the following claims. Further, any of the interface systems described herein may be interacted with through gestures and/or voice commands from a user. Utilizing voice commands promotes a sterile environment. Further, in some embodiments, the voice commands include one or more trigger words to allow the user to interact/manipulate the interface systems.

1. An interface system for ultrasound measurements, the system comprising:
a computer display configured for displaying the ultrasound measurements; and one or more processors programmed and configured to:
receive sets of ultrasound signals through a plurality of transducers of an ultrasound probe proximate to a lumen wall;
for each received ultrasound signal, calculate a distance between the receiving ultrasound transducer and the lumen;
based on the calculated distances for each set of ultrasound signals, determine a respective shape of a cross-section of the lumen; and
cause the computer display to simultaneously generate a plurality of representations of the lumen indicating differences in size and geometry between the respective shapes.

2. The interface system of claim 1, wherein the plurality of representations comprises two or more of the respective shapes overlapping each other from a front-facing perspective.

3. The interface system of claim 2, wherein each respective shape of a cross-section of the lumen represents a different longitudinal position of the lumen.

4. The interface system of claim 2, wherein a plurality of the respective shapes of a cross-section of the lumen represent a same longitudinal position of the lumen at different time points.

5. The interface system of claim 4, wherein a first time point of the different time points represents a time prior to a lumen-treatment procedure and a second time point of the different time points represents a time after the lumen-treatment procedure.

6. The interface system of claim 5, wherein the lumen-treatment procedure is at least one of a stent placement, angioplasty, or obstruction crossing procedure.

7. An interface system for ultrasound measurements, the system comprising:
a computer display configured for displaying the ultrasound measurements; and one or more processors programmed and configured to:
receive sets of ultrasound signals through a plurality of transducers of an ultrasound probe proximate to a lumen wall;
for each received ultrasound signal, calculate a distance between the receiving ultrasound transducer and the lumen;
based on the calculated distances for each set of ultrasound signals, determine a respective shape of a cross-section of the lumen;
receive image data representing the lumen wall, the image data distinct from the calculated distance measurements; and
cause the computer display to generate a representation of the respective shapes adjacent a representation of the image data.

8. The interface system of claim 7, wherein the image data representing the lumen wall comprises at least one of angiography, optical coherence tomography (OCT), or intravascular ultrasound (IVUS) image data.

9. The interface system of claim 7, wherein the image data is spatially co-registered with the respective shapes to co-align with corresponding regions of the lumen wall.

10. The interface system of claim 9, wherein a representation of the shapes comprises a longitudinal curve fit and wherein the representation of the image data comprises a longitudinal representation spatially co-registered with the longitudinal curve fit.

11. The interface system of claim 10, wherein the one or more processors are further programmed to:
cause the computer display to present an option for a user to select a longitudinal position within the longitudinal curve fit for generating a corresponding representation of a cross-sectional curve fit and co-registered cross-sectional image data.

12. A method for generating images of ultrasound measurements, the method comprising:
receiving distance measurements between a probe and a structure wall over different times and longitudinal positions of the structure wall, each measurement respectively based on an ultrasound signal from a transducer proximate to the structure wall;
determining a plurality of curve fits based on the distance measurements, the plurality of the curve fits each representing a cross-section of the structure; and
causing a computer display to generate a representation of the plurality of the curve fits overlapping each other and with respect to the different times or longitudinal positions.

13. A method for generating images of ultrasound measurements, the method comprising:
receiving sets of ultrasound signals through a plurality of transducers of an ultrasound probe proximate to a lumen wall;
for each received ultrasound signal, calculating a distance between the receiving ultrasound transducer and the lumen;
based on the calculated distances for each set of ultrasound signals, determining a respective shape of a cross-section of the lumen; and
simultaneously generating in a computer display a plurality of representations of the lumen indicating differences in size and geometry between the respective shapes.

14. The method of claim 13, wherein image data representing the lumen wall comprises at least one of angiography, optical coherence tomography (OCT), or intravascular ultrasound (IVUS) image data.

15. The method of claim 12, wherein a user interacts with the computer display through gestures and voice commands.

16. The method of claim 15, wherein use of the voice commands promote a sterile environment.

17. The method of claim 16, wherein the voice commands comprise one or more trigger words to allow the user to interact/manipulate the interface systems without breaking the sterile environment.

18. The interface system of claim 1, wherein the computer is configured to recognize a user interaction through gestures and voice commands.

19. The interface system of claim 18, wherein use of the voice commands promote a sterile environment.

20. The interface system of claim 19, wherein the voice commands comprise one or more trigger words to allow the user to interact/manipulate the interface systems without breaking the sterile environment.
